(19) United States
(12) Patent Application Publication
(10) Pub. No.: US 2005/0032912 A1
(43) Pub. Date: Feb. 10, 2005

(54) Anaesthetic compositions and method for their administration

(76) Inventor: Michael Ernest Garrett, Woking (GB)

Correspondence Address: FOLEY & LARDNER, P.O. BOX 80278, SAN DIEGO, CA, 92138-0278, US

(21) Appl. No.: 10/492,272

(22) PCT Filed: Oct. 8, 2002
(86) PCT No.: PCT/GB02/04574

(30) Foreign Application Priority Data
Oct. 8, 2001 (GB) 01240712

(51) Int. Cl. A61L 9/04; A61K 31/05; A61K 31/03

(52) U.S. Cl. 514/731; 424/45; 514/749

(57) ABSTRACT

An inhalation anaesthetic formulation comprising a suspension of the anaesthetic agent in aqueous solution.

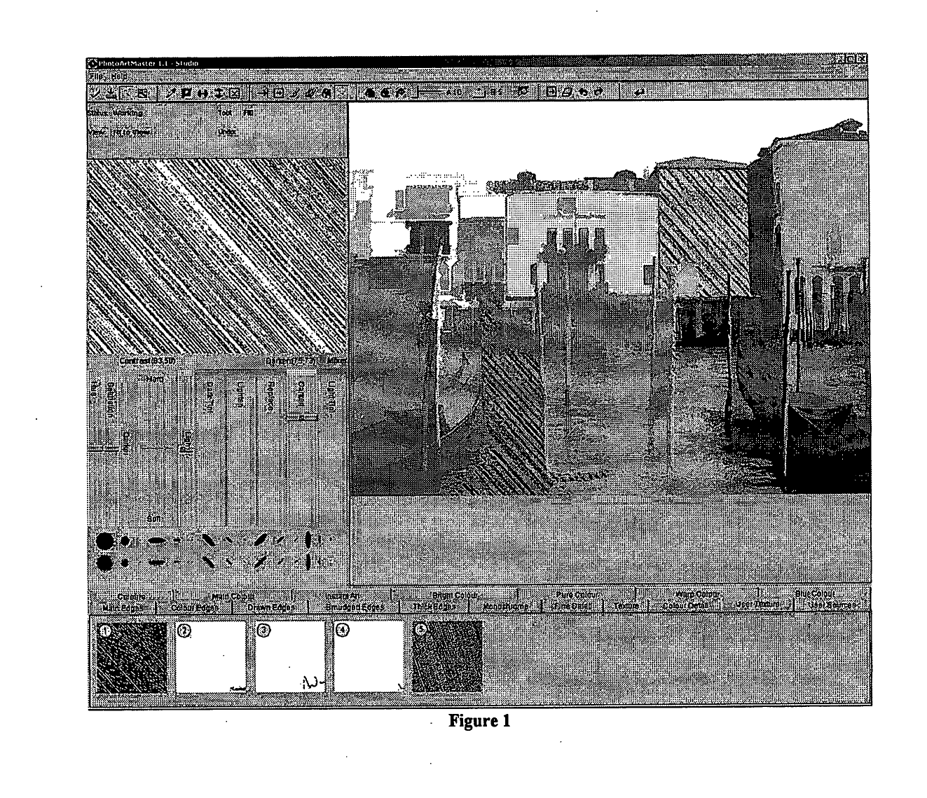

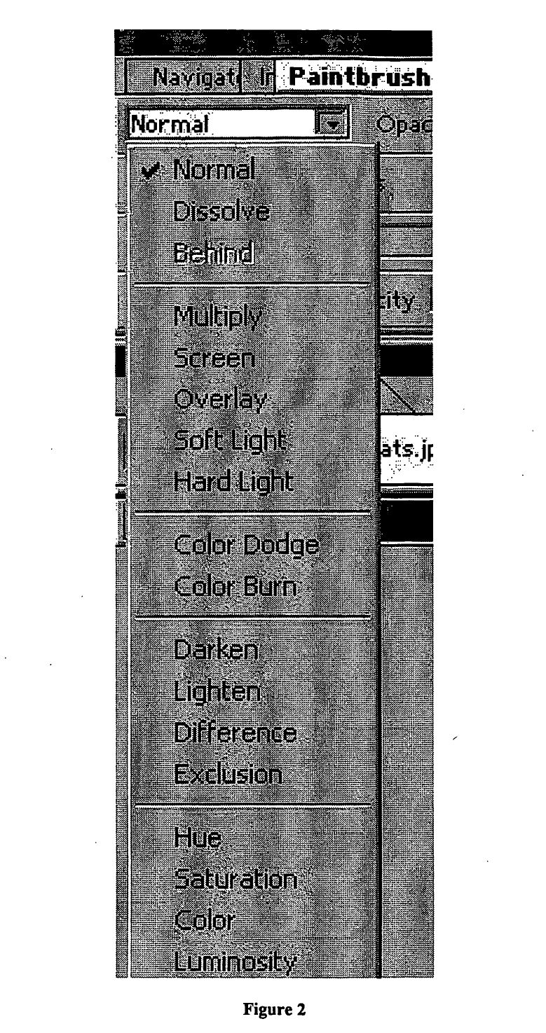

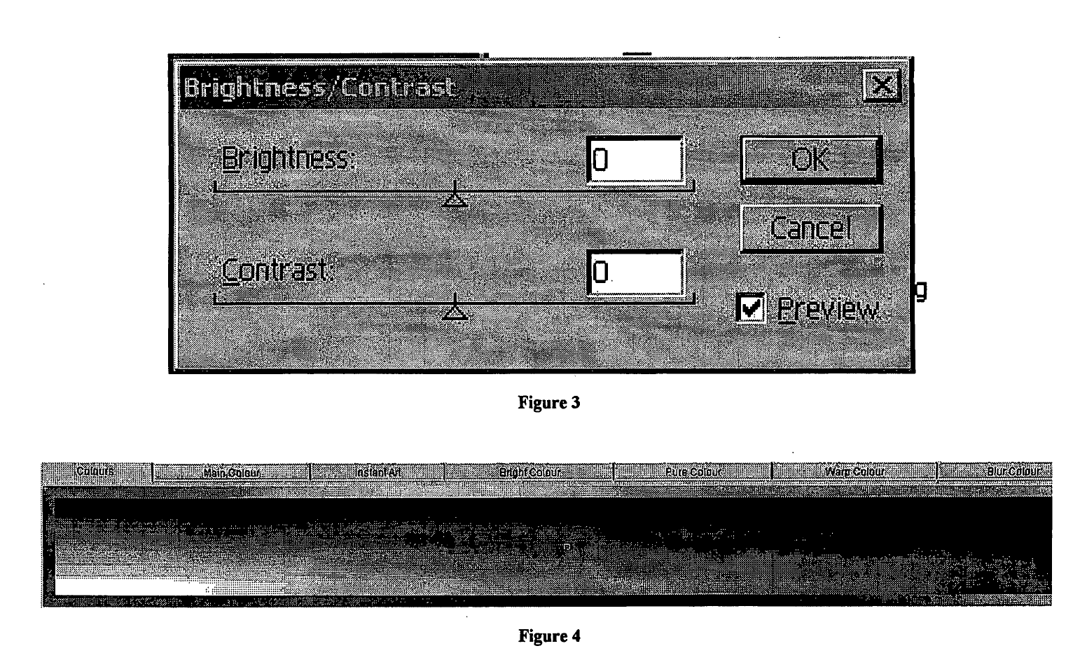

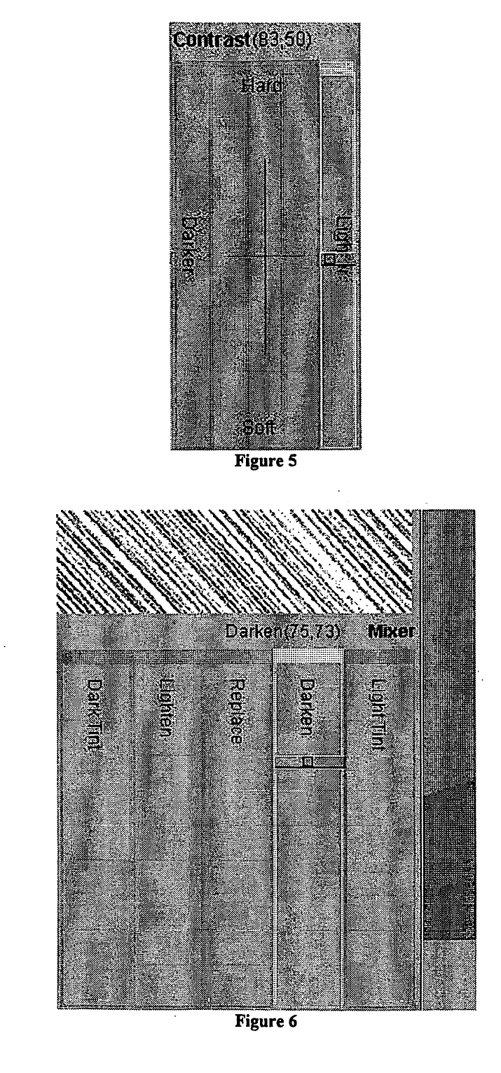

This invention relates to anaesthetic compositions and methods for their administration and, more particularly, to anaesthetic formulations which render them suitable for novel methods of administration.

Some of the best modern anaesthetics are administered to patients in vapour form by inhalation admixed with air, nitrogen and/or oxygen. Such anaesthetics include enflurane, isoflurane and desflurane. The anaesthetic agents themselves clearly need to be in the vapour form prior to inhalation and this is achieved by the use of a vaporiser—in reality a small heating apparatus—which, due tot their different boiling points, is specific to each anaesthetic agent.

The vaporiser is in turn connected to an anaesthesia delivery apparatus which mixes the anaesthetic agent to a concentration set by the anaesthetist in the gas stream to be inhaled by the patient.

There can, however, be problems associated with the administration of such inhaled anaesthetic agents. They tend to have a boiling point somewhat higher than ambient temperatures, for example 23.5° C. for desflurane, and they therefore require to be heated to produce a sufficient vapour pressure to provide the amount of anaesthetic required.

On particular problems is that as soon as the vaporised anaesthetic agent is mixed with the gas stream to be inhaled by the patient—usually at ambient temperatures—there is a tendency for the anaesthetic agent vapour to condense within the administration apparatus and delivery tubes to the patient. It is therefore difficult for the anaesthetist to be sure of the amount of anaesthetic agent being administered at any particular time. This problem is exacerbated by the fact that the anaesthetic agent concentrations in the gas stream being administered tend to be very low and there is therefore a fairly small margin of correctly administered dosages.

Other anaesthetic agents are administered to the patient by means of injection. Although generally less desirable than inhaled agents for reasons of ease of administration and the maintenance of a controlled amount of the anaesthetic composition, these are used in an variety of applications. For example, propofol is a widely used anaesthetic administered to the patient by injection. The anaesthetic agents is propofol contained in an appropriate formulation. Propofol is an oil at ambient temperature and is not readily soluble in water. Propofol must be thoroughly distributed in any anaesthetic formulation, and generally brought in to solution, because the administration of propofol itself in to the patient's blood stream can cause severe problems.

A number of different agents have been proposed over the years to promote the solubility of the propofol in aqueous (or other) media including propylene glycol and a variety of surfactants including various surfactants known as poloxamers which are sometimes known as “lutols” in Europe and “pluronics” in the US.

Poloxamers are generally polymeric surfactants and are composed of poly (a-oxyethylene-b-oxypropylene-a-oxyethylene) triblock copolymers. Although the properties of poloxamers differ widely, they are generally non-toxic.

The surfactant poloxamers act by virtue of their possessing hydrophilic and hydrophobic properties (or regions) and act to form “micelles” by encapsulating fine dispersions of oily or fatty molecules of suitably compatible composition with their hydrophilic properties acting outwardly, ie to water molecules of the “solution”, and their hydrophobic properties inwardly, ie in contact with the oily or fatty substance.

The overall effect of such a micellar structure is to solubilise the oily or fatty substance.

The possibility of using micellar polyoxamer preparations for propofol are disclosed in Patent Application Publication No. WO 01/64187 in the name of Maelor Pharmaceuticals Limited.

There remains a need to overcome problems associated with injectible anaesthetic agents and to provide formulation and methods for the administration of anaesthetic agents by inhalation.

The invention generally provides solutions to these problems through the provision of novel formulations and methods.

In accordance with the invention, there is provided an inhalation anaesthetic formulation comprising a suspension of the anaesthetic agent in water (aqueous solution).

The above named anaesthetic agents—enflurane, ethrane, desflurane and propofol—can be regarded as oils or oily substances and the formulations of the invention are therefore formed by suspending the oil in water itself or an aqueous solution.

It is important that the droplets of oily anaesthetic agent forming the suspension are very small such that they do not coalesce. Advantageously, the droplets are sub-micron (μm) in size.

The anaesthetic agents can be present in any suitable concentration in the aqueous solution as determined by the anaesthetist. Propofol in particular is advantageously provided at a concentration of 0.5% to 2% (w/w), most advantageously about 1%, for normal anaesthetic use although 2% or more may be employed for long term analgesia. Dosage for the induction of anaesthesia is typically 2.5 mg/kg patient bodyweight and the half-life in the bloodstream is of the order of nine hours.

In their simplest form, the anaesthetic formulation of the invention may be passed in to a nebuliser which renders the suspension in to a very fine stream of water (aqueous) droplets with which the oil droplets are admixed and passed to the patient for inhalation by general means known per se. In effecting this, the carrier gas for the formulation may be air or other relevant gas or gas mixture.

Alternatively, the required small droplets can be achieved by other conventional techniques including ultrasonics, mechanical agitation or high shear methods. The resulting suspension may then be administered to a patient by means of any suitable inhalation apparatus.

The presence of water in the inhaled anaesthetic is generally helpful to the patient and preferably some further means of humidifying the inhaled anaesthetic is provided. The “mist” of the anaesthetic agent and water is inhaled by the patient and the droplets of oil anaesthetic become attached to the surface of the patient's lungs and are then absorbed in to the blood stream.

In preferred embodiments of the invention, one or more co-solvents and/or surfactants may be employed in the formulation of the invention to fully or partially solubilise the anaesthetic agent in the water/aqueous solution. The resulting formulations in which the anaesthetic agent is releasable therefrom are included in the meaning of the term “suspension” used herein.

Water-misible co-solvents which may be considered include propylene glycol, glycerol and ethylene/polyethylene oxides.

Surfactants which may be considered include various micellar solubilisation agents.

In the case of propofol in particular, the surfactant is preferably a poloxamer which is pharmaceutically acceptable for an inhaled formulation. As stated above, poloxamers promote the formation of a micellar structure with the propofol.

Poloxamers vary greatly in their constituent make up, and are generally characterised by a ratio of polyoxyethylene (PEO) units to polyoxypropylene oxide (PPO) units, and the molecular weight of the propylene oxide block. Within the general range of poloxamers available, it has generally been found that those having an average molecular weight of propylene oxide of greater than about 1500 Dalton (D) and an average percent ethylene oxide of greater than about 30% w/w are suitable. More preferably, the PPO portion is at least 2000 D while the EO portion is at least 40% w/w.

Where formulations of the invention comprise a single poloxamer, these preferably contain at least 0.8% w/w propofol, with formulations containing 1% w/w being more preferred. The upper end of the range is generally dictated by the ability of the system to support higher concentrations of propofol. With concentrations of 10% w/w poloxamer in water, the maximum concentration of propofol is about 3.2% when a poloxamer such as P237 is used. Poloxamer combinations can take this even higher. However, a physiologically effective concentration is 1%, so that higher concentrations result in smaller volumes being required which can be awkward to administer. Thus, a propofol concentration in the range of 1% to 1.5% w/w is preferred.

Individually preferred poloxamers are P234, P237, P338 and P407. P407 is particularly preferred as, although it dissolves 1.7% propofol, it has been approved for medicinal purposes. P234 and P338 are better than P407, but neither has been approved. Likewise, P237 provides excellent uptake, but also has yet to be approved.

Combinations of poloxamers may be employed in the invention. Such combinations tend to act in a synergistic manner.

As noted above, poloxamers comprise PPO units and EO units. The PPO units are generally hydrophobic, and form the central portion of any micelle. In micelles with only one poloxamer, PPO blocks align with each other, while EO blocks also align with each other on the outside, to form a thermodynamically stable system. In a mixed micelle, with poloxamers of differing PPO length, when the PPO blocks of different poloxamers align, either a “hole” is left in the micellar interior, or part of the EO block of the shorter poloxamer must align with the PPO of the larger molecule.

The presence of propofol encourages the formation of mixed micelles. Propofol tends to compensate for the difference in PPO length by occupying the space at the end of the shorter PPO chain.

Preparation of anaesthetic formulations of the invention is generally straightforward. Although the constituents of the formulations can be added in any sequence, as desired, it will be appreciated that propofol in particular is virtually insoluble in water, so that the most expedient method of mixing is to prepare a poloxamer solution in water, followed by the addition of propofol.

Poloxamer P407 is readily soluble in water, but heating of the water and the poloxamer, whilst mixing, can generally increase the speed of micelle formation. In addition, some poloxamers require increased temperatures in order to satisfactorily micellise in water. In general, concentrations of poloxamer of about 10% w/v are useful in the present invention, but concentrations of poloxamers, whether single or mixed, can be selected as appropriate and will generally be above 0.5% and below about 20%. More preferred concentrations are from 5% to 15%. Some poloxamers will begin to gel at higher concentrations, and any poloxamer concentration that gels at body temperature, especially when in association with propofol, should be avoided for injection purposes.

One reported after-effect of propofol anaesthesia is the reduction in tremors for patients who have Parkinson's disease. The effect can last for up to three days. At this time the concentration in the body will have fallen to less than 0.02 mg/kg bodyweight, far below that required for anaesthesia. Low dosages such as these could be maintained, if desired, by an inhalation spray of water based propofol with a much lower concentration of the active ingredient. For example a 0.01% concentration which would be too low for misuse as an anaesthetic, but would be sufficiently strong to achieve the low doses reportedly effective in controlling muscle tremors.

The inhaled aqueous formulations of the invention are particularly useful for the maintenance of this effect in respect of Parkinson's disease.

In use of the invention, the anaesthetic formulation should be available in a form in which the anaesthetic agent is present at a concentration of delivery direct to the patient by means of a nebuliser (or whatever).

Alternatively, the formulation may be made available in a form in which the anaesthetic agent is present in a concentration form such that dilution may be effected by the anaesthetist at the time of administration.

Overall, the formulations of the invention can in general be administered without the problem of condensation of the anaesthetic agent occurring within the administration apparatus and/or delivery tubes to the patient.

1. An inhalation anaesthetic formulation comprising a suspension of the anaesthetic agent in aqueous medium.

2. A formulation according to claim 1 in which the anaesthetic agent is selected from the group consisting of enflurane, ethrane, desflurane, propofol, and mixtures of any two or more thereof.

3. A formulation according to claim 1 in which the droplets of anaesthetic agent forming the suspension are very small such that they do not coalesce.

4. A formulation according to claim 3 in which the droplets are sub-micron in size.

5. A formulation according to claim 1 in which the anaesthetic agent is present at a concentration of 0.5% to 2% (w/w).

6. A formulation according to claim 1 in which administration of the formulation is effected by means of a nebuliser.

7. A formulation according to claim 1, further comprising one or more co-solvents and/or surfactants.

8. A formulation according to claim 7 wherein said co-solvent is selected from the group consisting of propylene glycol, glycerol, ethylene/polyethylene oxide, and mixtures of any two or more thereof.

9. A formulation according to claim 7 wherein said surfactant is a micellar solubilisation agent.

10. A formulation according to claim 9 wherein said surfactant is a poloxamer.

11. A formulation according to claim 2 in which the droplets of anaesthetic agent forming the suspension are very small such that they do not coalesce.

12. A formulation according to claim 11 in which the droplets are sub-micron in size.

13. A formulation according to claim 2 in which the anaesthetic agent is propofol present at a concentration of 0.5% to 2% (w/w).

14. A formulation according to claim 2 in which administration of the formulation is effected by means of a nebuliser.

15. A formulation according to claim 2, further comprising one or more co-solvents and/or surfactants.

16. A formulation according to claim 15 wherein said co-solvent is selected from the group consisting of propylene glycol, glycerol, ethylene/polyethylene oxide, and mixtures of any two or more thereof.

17. A formulation according to claim 15 wherein said surfactant is a micellar solubilisation agent.

18. A formulation according to claim 17 wherein said surfactant is a poloxamer.

19. A method for administration of an anaesthetic to a subject by inhalation, said method comprising:
passing the formulation of claim 1 in to a nebulizer, and thereafter
passing said formulation to the patient for inhalation.

20. A method for administration of an anaesthetic to a subject by inhalation, said method comprising passing the formulation of claim 1 to the patient for inhalation after passing said formulation through a nebulizer.
